SEQUENCE LISTING (78 sequences)

SEQ ID NO: 1 - DNA, 19 bp, artificial sequence
  Probe
atctaaacatagcctcgct

SEQ ID NO: 2 - DNA, 18 bp, artificial sequence
  Probe
atcaatggatacgctgcc

SEQ ID NO: 3 - DNA, 18 bp, artificial sequence
  Probe
atctagatgagcgcatgg

SEQ ID NO: 4 - DNA, 18 bp, artificial sequence
  Probe
atctaacaagcctcgctc

SEQ ID NO: 5 - DNA, 18 bp, artificial sequence
  Probe
atctagcacgcaagagga

SEQ ID NO: 6 - DNA, 18 bp, artificial sequence
  Probe
atcaaatggatgcgttgc

SEQ ID NO: 7 - DNA, 18 bp, artificial sequence
  Probe
tgtcttggactcgtccaa

SEQ ID NO: 8 - DNA, 18 bp, artificial sequence
  Probe
atcgaaagatggtgcaca

SEQ ID NO: 9 - DNA, 20 bp, artificial sequence
  Probe
atcaaaatgtatgcgttgtc

SEQ ID NO: 10 - DNA, 19 bp, artificial sequence
  Probe
atctagatgagcgcatagt

SEQ ID NO: 11 - DNA, 19 bp, artificial sequence
  Probe
atcaaatggatgcgttgcc

SEQ ID NO: 12 - DNA, 18 bp, artificial sequence
  Probe
aacactcgggctctgttc

SEQ ID NO: 13 - DNA, 18 bp, artificial sequence
  Probe
atcaattggatgcgctgc

SEQ ID NO: 14 - DNA, 18 bp, artificial sequence
  Probe
atctctgttggtttcggg

SEQ ID NO: 15 - DNA, 20 bp, artificial sequence
  Probe
atctgatacttgatgctcct

SEQ ID NO: 16 - DNA, 19 bp, artificial sequence
  Probe
atctaaacggatgctttgc

SEQ ID NO: 17 - DNA, 20 bp, artificial sequence
  Probe
atctagttcgtaagagtgtg

SEQ ID NO: 18 - DNA, 18 bp, artificial sequence
  Probe
actcaggcttggccagag

SEQ ID NO: 19 - DNA, 21 bp, artificial sequence
  Probe
atctaacaagcagatttttgg

SEQ ID NO: 20 - DNA, 20 bp, artificial sequence
  Probe
atctagcagatgagatctct

SEQ ID NO: 21 - DNA, 18 bp, artificial sequence
  Probe
atctgaatgcacagcgct

SEQ ID NO: 22 - DNA, 18 bp, artificial sequence
  Probe
atctggcaaagactgtgg

SEQ ID NO: 23 - DNA, 17 bp, artificial sequence
  Probe
accctgcttggtggtgg

SEQ ID NO: 24 - DNA, 17 bp, artificial sequence
  Probe
aggtgttgtcccaccgc

SEQ ID NO: 25 - DNA, 17 bp, artificial sequence
  Probe
cctccatcttggtggtg

SEQ ID NO: 26 - DNA, 16 bp, artificial sequence
  Probe
aggtgttgtcccaccg

SEQ ID NO: 27 - DNA, 16 bp, artificial sequence
  Probe
accctgcttggtggtg

SEQ ID NO: 28 - DNA, 18 bp, artificial sequence
  Probe
cggtgtctgttgttgctc

SEQ ID NO: 29 - DNA, 18 bp, artificial sequence
  Probe
agagtgttgtcccaccat

SEQ ID NO: 30 - DNA, 17 bp, artificial sequence
  Probe
gggtcggtgtgttgttg

SEQ ID NO: 31 - DNA, 16 bp, artificial sequence
  Probe
gggtgctgtcccccca

SEQ ID NO: 32 - DNA, 18 bp, artificial sequence
  Probe
cctccatcttggtggtgg

SEQ ID NO: 33 - DNA, 18 bp, artificial sequence
  Probe
agagttgtcccaccatct

SEQ ID NO: 34 - DNA, 18 bp, artificial sequence
  Probe
gggatgttgtcccaccat

SEQ ID NO: 35 - DNA, 16 bp, artificial sequence
  Probe
gggtgccggtgtgttg

SEQ ID NO: 36 - DNA, 17 bp, artificial sequence
  Probe
tgagtggtgtccctcca

SEQ ID NO: 37 - DNA, 17 bp, artificial sequence
  Probe
ggcgtgttgttgccctg

SEQ ID NO: 38 - DNA, 17 bp, artificial sequence
  Probe
gagctgttgtcccacca

SEQ ID NO: 39 - DNA, 17 bp, artificial sequence
  Probe
gcctcacacttggtggt

SEQ ID NO: 40 - DNA, 18 bp, artificial sequence
  Probe
tcaccggttttggtgtgg

SEQ ID NO: 41 - DNA, 16 bp, artificial sequence
  Probe
ggtgttgtcccaccgc

SEQ ID NO: 42 - DNA, 17 bp, artificial sequence
  Probe
gggtcgggcgtgttgtt

SEQ ID NO: 43 - DNA, 16 bp, artificial sequence
  Probe
tggtggcggggtgtgg

SEQ ID NO: 44 - DNA, 23 bp, artificial sequence
  Probe
atcacctcctttctaaggagcac

SEQ ID NO: 45 - DNA, 21 bp, artificial sequence
  Probe
atggaatgtcgcaaccaaatg

SEQ ID NO: 46 - DNA, 21 bp, artificial sequence
  Probe
atagaatgtcgcaaccaaatg

SEQ ID NO: 47 - DNA, 16 bp, artificial sequence
  Probe
actaccacccgcaggc

SEQ ID NO: 48 - DNA, 18 bp, artificial sequence
  Probe
actaccacccgcactgcg

SEQ ID NO: 49 - DNA, 19 bp, artificial sequence
  Probe
gcgacgcgtcgatctacag

SEQ ID NO: 50 - DNA, 19 bp, artificial sequence
  Probe
ggcgacgtgtcgatctacg

SEQ ID NO: 51 - DNA, 16 bp, artificial sequence
  Probe
gcgacgcgtcgatcta

SEQ ID NO: 52 - DNA, 20 bp, artificial sequence
  Probe
atctagcacagcctggtgcg

SEQ ID NO: 53 - DNA, 21 bp, artificial sequence
  Probe
atctactacagcctggtgcgc

SEQ ID NO: 54 - DNA, 20 bp, artificial sequence
  Probe
tctaccacagcctggtgcgc

SEQ ID NO: 55 - DNA, 19 bp, artificial sequence
  Probe
ctacaacagcctggtgcgc

SEQ ID NO: 56 - DNA, 18 bp, artificial sequence
  Probe
cgatctacggcagcctgg

SEQ ID NO: 57 - DNA, 18 bp, artificial sequence
  Probe
cgatctacgccagcctgg

SEQ ID NO: 58 - DNA, 21 bp, artificial sequence
  Probe
tcgatctacgacaccctggtg

SEQ ID NO: 59 - DNA, 19 bp, artificial sequence
  Probe
ctacatcctgggcatggcc

SEQ ID NO: 60 - DNA, 18 bp, artificial sequence
  Probe
tacatcctgggcctggcc

SEQ ID NO: 61 - DNA, 16 bp, artificial sequence
  Probe
catcctgggcgtggcc

SEQ ID NO: 62 - DNA, 17 bp, artificial sequence
  Probe
gggcatagcccgagtcg

SEQ ID NO: 63 - DNA, 17 bp, artificial sequence
  Probe
gggcattgcccgagtcg

SEQ ID NO: 64 - DNA, 16 bp, artificial sequence
  Probe
gggcatcgcccgagtc

SEQ ID NO: 65 - DNA, 19 bp, artificial sequence
  Probe
acccgggtgacgacttctc

SEQ ID NO: 66 - DNA, 19 bp, artificial sequence
  Probe
acccgggtgaccacttctc

SEQ ID NO: 67 - DNA, 21 bp, artificial sequence
  Primer
tgggacgaagtcgtaacaagg

SEQ ID NO: 68 - DNA, 18 bp, artificial sequence
  Primer
tgccaaggcatccaccat

SEQ ID NO: 69 - DNA, 16 bp, artificial sequence
  Primer
acgttcccgggccttg

SEQ ID NO: 70 - DNA, 18 bp, artificial sequence
  Primer
tgacagctcccccgaggc

SEQ ID NO: 71 - DNA, 18 bp, artificial sequence
  Primer
ggctcatatcgcgaatgc

SEQ ID NO: 72 - DNA, 19 bp, artificial sequence
  Primer
ctcatcatcaaagcgggac

SEQ ID NO: 73 - DNA, 22 bp, artificial sequence
  Primer
cgggtgctctatgcaatggttc

SEQ ID NO: 74 - DNA, 20 bp, artificial sequence
  Primer
tcctcgtcgatttccctcag

SEQ ID NO: 75 - DNA, 17 bp, artificial sequence
  Primer
tggcgcaccttcaccct

SEQ ID NO: 76 - DNA, 23 bp, artificial sequence
  Primer
gaaccagcggaaaatagttggac

SEQ ID NO: 77 - DNA, 19 bp, artificial sequence
  Primer
gacttctgcgagggtggct

SEQ ID NO: 78 - DNA, 19 bp, artificial sequence
  Primer
tacgctccggtgtaggcac

(19) United States
(12) Patent Application Publication
(10) Pub. No.: US 2005/0136432 A1
(43) Pub. Date: Jun. 23, 2005

(54) Mycobacterial disease detection chip and fabrication method thereof and method of detecting mycobacterial disease and primer set for mycobacterial disease and drug resistance detection

(76) Inventors: Kun-Shan Lu, Taipei (TW); Cheng-Yu Wei, Hualien City (TW); Yu-Jie Zhao, Shenyang City (TW)

Correspondence Address: J C PATENTS, INC., 4 VENTURE, SUITE 250, IRVINE, CA, 92618, US

(21) Appl. No.: 10/911,171

(22) Filed: Aug. 3, 2004

(30) Foreign Application Priority Data
Dec. 19, 2003 (TW) 92136102

(51) Int. Cl. C12Q 1/68; G01N 33/53; C12M 1/34

(52) U.S. Cl. 435/6; 435/287.2; 427/2.11

(57) ABSTRACT

A mycobacterial disease microarray detection chip includes a plurality of probes immobilized on a matrix, wherein each of the probe is selected from the group of deoxyribonucleotide sequences depicted in the SEQ ID NOs. 1 to 66. Since these probes are formed with deoxyribonucleotide sequences specific to mycobacterial disease, they can be used to detect whether the patient has contracted mycobacterial disease and the mycobacterial disease pathogen infected the patient has drug resistance.

### CROSS-REFERENCE TO RELATED APPLICATION

This application claims the priority benefit of Taiwan application serial no. 92136102, filed on Dec. 19, 2003.

### BACKGROUND OF THE INVENTION

1. Field of the Invention

The present invention relates to a detection device for an illness and a fabrication method thereof, a method for detecting the illness and a primer set for detecting the illness. More particularly, the present invention relates to a mycobacterial disease detection chip and a fabrication method thereof, a method for detecting mycobacterial disease and a primer set for mycobacterial disease and drug resistance detection.

2. Description of Related Art

Mycobacterial disease, caused by the pathogens Mycobacterium spp, is one of the most common human disease at present. Although vaccination is available for preventing mycobacterial disease, it is still common in the out-country or undeveloped regions. Therefore, one of important task in the medicine science is the prevention of mycobacterial disease. Based on the mycobacterial disease statistics reported by Center for Disease Control of the Department of Health (CDCDH), more than 15 thousands new cases are reported each year, about 70% of the total announced number of infectious diseases. From the statistics for the death of infectious diseases, the persons died of mycobacterial disease are about 15 hundreds in one year, five times of the other infectious diseases. Therefore, mycobacterial disease is considered the most serious infectious disease in Taiwan, or called the top killer of the infectious diseases in Taiwan. In the Year 2001 annual report of CDCDH for mycobacterial disease prevention, it is indicated that a total number of persons died of mycobacterial disease in Taiwan was 1,747, the men death roll is 3.42 times of the woman death roll, and the men death rate is 3.27 times of the woman death rate. Compared with the past, mortality of mycobacterial disease for men is increasing nowadays. In addition, mortality of mycobacterial disease becomes higher as the age increases. Between the 1,299 cases of mycobacterial disease death, 77.3% (1,004 persons) of the patients are aged persons older than 65 years old. At the present time, the age distribution of mycobacterial disease death is shifted to the aged population.

Because the treatment of mycobacterial disease includes lengthy medication, one serious problems encountered in the clinical treatments of mycobacterial disease is the drug resistant Mycobacterium spp. Especially, more and more reports regarding the drug resistant Mycobacterium spp. come out after 90s. Additionally, certain gene mutations of the Mycobacterium spp. are reported to be highly related to their drug resistance. Therefore, for mycobacterial disease of diversified pathogens, a prompt and accurate diagnosis is one vital factor for treating mycobacterial disease and avoiding misspending the medical resources.

The diagnosis of mycobacterial disease mainly relies on examining the existence of Mycobacterium spp incubated or isolated from the secretions or tissues of the patient. The existing examination methods include stain assays, Mycobacterium spp. culture or radio-immunological assays. Although the stain diagnostic assay is simple and fast by using a microscope, a substantial amount of the bacteria is required along with particular staining techniques. Although bacterial (Mycobacterium spp) incubation culture is highly sensitive, this test requires a long time to produce meaningful results. The radio-immunological assay is rapid, but has an inferior sensitivity.

Microarray detection chip is one of the most important technological advancements in the high-tech field in recent years. Microarray detection chip is a high technology product that bases on a multidisciplinary effort from areas of physics, chemistry, microelectronics, precision machining and bioscience. Mircroarray detection chip can provide a large amount of probes with specific DNA sequences immobilized on a matrix, and a great deal of information is produced after the probe is reacted with a test sample (for example, DNA). Therefore, a microarray detection chip can be used for screening diseases. However, for different types of diseases, a great deal of effort must be devoted to the fabrication of the microarray detection chip in order to design a specific probe and a primer set to amplify the DNA fragment of the test sample of the patient. The current mycobacterial disease detection techniques still have not included a microarray detection chip for screening mycobacterial disease.

### SUMMARY OF THE INVENTION

Accordingly, the present invention provides a specific probe designed according to the general and drug resistant pathogens of mycobacterial disease and a corresponding primer set, and using this microarray detection chip technique to develop an accurate and rapid mycobacterial disease detection chip and a fabrication method thereof, and a detection method.

In accordance to the present invention, a fabrication method for a mycobacterial disease detection chip is provided, wherein the resulting detection chip can concurrently screen a plurality of mycobacterial disease pathogens.

The present invention also provides a microarray detection chip for detecting whether the patient has mycobacterial disease and the pathogen of mycobacterial disease is drug resistant.

The present invention further provides a detection method for mycobacterial disease for accurately and rapidly detecting whether a patient has contracted mycobacterial disease and the pathogen of mycobacterial disease is drug resistant.

The present invention further provides a primer set for mycobacterial disease and drug, resistance detection, for amplifying a specific deoxyribonucleic acid (DNA) fragment of a patient's sample.

The present invention provides a fabrication method for a mycobacterial disease detection chip, wherein the method includes designing a plurality of probe sequences and these probe sequences includes at least DNA sequences depicted in SEQ ID NOs. (Sequence Identifier Number) 1 to SEQ ID No. 44. The step of designing the probe sequences further includes designing a plurality of primer sets that corresponds to the probe sequences in order to amplify specific DNA fragments of the test sample from the patient. The primer sets comprises at least 2 primer sets that are formed with the DNA sequence depicted in the SEQ ID NOs. 67 to 70. A probe synthesis step is conducted to synthesize the probes formed with DNA sequences depicted in SEQ ID NOs. 1 to SEQ ID NO. 44. Thereafter, a spotting step is performed to spot respectively these probes on a matrix. Moreover, the step of designing the probe sequences further comprises designing a plurality of drug-resistance analysis probe sequences and/or a plurality of quality control probe sequences. The probe synthesis step and the spotting step are also performed to these drug-resistance analysis probe sequences and quality control probe sequences. The drug-resistance analysis probe sequences includes at least the DNA sequences depicted in the SEQ ID NOs. 45 to 66. Further, the step of designing the drug-resistance analysis probe sequences further comprises designing a plurality of primer sets that corresponds to the drug-resistance analysis probe sequences. The primer sets corresponding to the drug-resistance analysis probe sequences includes at least 4 primer sets formed with the DNA sequences depicted in the SEQ ID NOs. 71 to 78.

The present invention provides a microarray detection chip for mycobacterial disease. This microarray detection chip for mycobacterial disease includes a plurality of probes immobilized on a matrix, wherein each probe is selected from the group consisting of the DNA sequences depicted in the SEQ ID NOs. 1-44. This microarray detection chip for mycobacterial disease includes a plurality of drug-resistance analysis probes and quality control probes. The drug-resistance analysis probes are applicable for detecting whether the pathogen infected the patient is drug resistant and each drug-resistance analysis probes is selected from the group consisting of the DNA sequences depicted in the SEQ ID NOs. 45 to 66.

The present invention provides a detection method for mycobacterial disease. This detection method includes providing the aforementioned microarray detection chip for mycobacterial disease. After treating a sample from the patient and obtaining the DNA from the sample, a plurality primer sets is used to conduct a polymerase chain reaction (PCR) to amplify a specific DNA fragment and to obtain a PCR product. The primers sets used in the PCR are selected from the 2 primer sets, which are formed with the DNA sequences depicted in the SEQ ID NO. 67-70. A hybridization procedure is then conducted to this PCR product, so that the PCR product reacts with the probes on the microarray detection chip. Thereafter, the test result from the microarray detection chip is analyzed. Especially, the detection method further comprises the drug-resistance analysis procedure. The drug-resistance analysis step includes immobilizing a plurality of drug-resistance analysis probes on the microarray detection chip. Each of the drug-resistance analysis probes is selected from the group consisting of the DNA sequences depicted in the SEQ ID NO. 45-66 and the primer sets used in the PCR includes 4 primer sets formed with the DNA sequences depicted in the SEQ ID NO. 71-78.

Since the microarray detection chip of the present invention employs the DNA sequence specific to mycobacterial disease, this detection chip can be used to detect whether a patient has mycobacterial disease and the mycobacterial disease pathogen infected the patient is drug-resistant, as references for the treatments of mycobacterial disease.

The present invention provides a primer set for detecting mycobacterial disease, wherein this primer set is selected from 6 primer sets that are formed with the DNA sequences depicted in the SEQ ID NOs. 67-78.

Since these primer sets (SEQ ID NOs. 67-78) are related to the specific pathogens of mycobacterial disease, these primer sets can be used to amplify the specific DNA fragment(s) of the patient and the detection method is applicable for detecting whether the patient has mycobacterial disease and the mycobacterial disease pathogen infected the patient is drug resistant.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a flow diagram illustrating the fabrication for a mycobacterial disease detection chip according to one embodiment of the invention.

FIG. 2 is a flow diagram illustrating the method of detecting mycobacterial disease according to one embodiment of the invention.

### DESCRIPTION OF THE EMBODIMENTS

The important aspects in the fabrication of a microarray detection chip are the designs of the probes and the primers set for amplifying the DNA segments of a patient's sample. The microarray detection chip can provide accurate information only with the design of specific probes and specific primer sets. The fabrication of the microarray detection chip for mycobacterial disease and the related detection method of the present invention are based on the aforementioned concepts. Although the disclosure herein refers to certain illustrated embodiments, it is to be understood that these embodiments are presented by way of example and not by way of limitation.

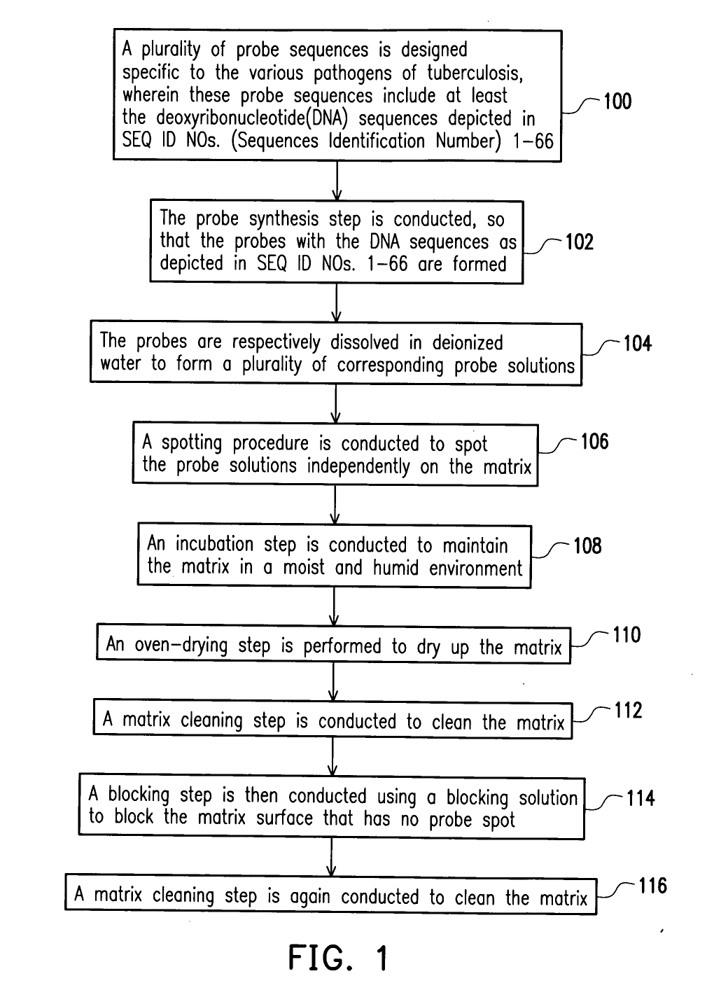

FIG. 1 is a flow diagram illustrating the fabrication method of a mycobacterial disease detection chip according to one embodiment of the invention.

Referring to FIG. 1, a plurality of probe sequences is designed specific to the various pathogens of mycobacterial disease, wherein these probe sequences include at least the deoxyribonucleotide sequences depicted in SEQ ID NOs. (Sequence Identification Number) 1-66 (step 100). These probe sequences, for example, are formed with 15 to 25 deoxyribonucleic acids. These probe sequences are designed according to the available gene sequences of the pathogens of mycobacterial disease obtained from a gene bank. Additionally, the DNA sequences depicted in the SEQ ID NOs. 1-44 are related to the pathogens of mycobacterial disease, while the DNA sequences depicted in the SEQ ID NOs. 45 to 66 are related to the drug-resistant pathogens of mycobacterial disease.

It is worth to note that the typical pathogens of mycobacterial disease include various strains of Mycobacterium spp. Table 1 lists the pathogens of mycobacterial disease and the corresponding SEQ ID NOs. of the probe sequences.

TABLE 1

| | SEQ ID NO. of the |
|---|---|
| Pathogens of mycobacterial disease | probe sequences |
| Abscessus | 1-23 |
| Africanum | 2-24 |
| Avium | 3-25 |
| Bovis | 2-26 |
| Chelonae | 4-27 |
| Diernhoferi | 5-28 |
| Gastri | 6-7-29 |
| Gilvum | 8-30 |
| Gordonae | 9-31 |
| Intracellulare | 10-32 |
| Kansasii | 11-12-33 |
| Marinum | 13-14-34 |
| Phlei | 15-35 |
| Scrofulaceum | 16-36 |
| Smegmatis | 17-37 |
| Szulgai | 13-18-38 |
| Terrae | 19-39 |
| Triviale | 20-40 |
| Tuberculusis | 2-41 |
| Vaccae | 21-42 |
| Xenopi | 22-43 |
| Fortuitum | 44 |

From the above table, if one pathogen corresponds to more than one probe sequences, it means that these probe sequences are the basis for mutually detecting the pathogen. For example, the probe sequences of the SEQ ID NOs. 1 and 23 are the basis for discriminating the abscessus pathogen. Further, if one pathogen corresponds to only one probe sequence, it means that this probe sequence is the basis for detecting the pathogen. For example, the probe sequence of the SEQ ID NO. 44 is basis for detecting the fortuitum pathogen.

In addition, some pathogens of mycobacterial disease are resistant to the drugs Ethambutol (EMB), Isoniazid (INH), Rifampin (RFP), Quinolones, Ciprofloxacin or Pyrazinamide (PZA), thus having drug resistance. Therefore, the present invention further design drug-resistance analysis probe sequences (sequences depicted in the SEQ ID NOs. 45 to 66) to detect whether the mycobacterial disease pathogen infected the patient is drug resistant. Table 2 lists the types of drug resistance and the corresponding

TABLE 2

| | SEQ ID NOs. of the |
|---|---|
| Drug resistance (resistant to) | probe sequences |
| EMB - INH - RFP | 45-46 |
| Quinolones - Ciprofloxacin | 47-58 |
| EMB | 59-64 |
| PZA | 65-66 |

As shown in Table 2, the drug resistance of the mycobacterial disease pathogens can be classified through the probe sequences of SEQ ID NOs. 45-66. For example, if positive results are present for the probe sequences of SEQ ID NOs. 45 and 46 in the subsequent testing procedures, the mycobacterial disease pathogen infected the patient has drug resistance to the drugs EMB, INH or RFP. Simultaneously, from the results toward the probe sequences of SEQ ID NOs. 59 and 64, it is possible to identify whether the mycobacterial disease pathogen infected the patient has drug resistance to EMB. Moreover, the wild type pathogen can be detected by using the probe sequences of SEQ ID NOs. 47-66 as the basis.

During the step of designing the probe sequence (step 100), the design of the primer sets that correspond to these probe sequences and drug-resistant analysis probe sequences, which are used to amplify the specific DNA sequence of the patient's sample, are also conducted. These primer sets corresponding to the probe sequences include at least 2 primer sets, which are formed with the DNA sequences depicted in the SEQ ID NOs. 67-70. These primer sets corresponding to the drug-resistant analysis probe sequences include at least 4 primer sets, which are formed with the DNA sequences depicted in the SEQ ID NOs. 71-78. Similarly, the design of these primer sets relies on the reliable gene sequences of the mycobacterial disease pathogens obtained from the gene bank.

Each primer set includes a 5′ to 3′ forward primer and a 3′ to 5′ reverse primer for amplifying the specific DNA fragments of the pathogens of mycobacterial disease. The following Table 3 lists the mycobacterial disease pathogens and the SEQ ID NOs. of the corresponding primer sets, while Table 4 lists the type of drug resistance for drug-resistant mycobacterial disease pathogens and the SEQ ID NOs. of the corresponding primer sets.

TABLE 3

SEQ ID NO. of the Primer
Set

| | Forward | Reverse |
|---|---|---|
| Primer sets | primer | primer |
| First | 67 | 68 |
| Second | 69 | 70 |

The DNA sequences depicted in the SEQ ID NOs. 67-70 constitute two primer sets. According to one embodiment, the first primer sets are used to conduct the first PCR and then the second primer sets are used to conduct the second PCR, for the 16S-18S gene sequences of the typical pathogens.

TABLE 4

SEQ ID NO. of the
Primer Set

| | Forward | Reverse |
|---|---|---|
| Drug resistance type | primer | primer |
| EMB - INH - RFP | 71 | 72 |
| Quinolone - ciprofloxacin | 73 | 74 |
| EMB | 75 | 76 |
| PZA | 78 | 78 |

Not only the four primer sets listed in Table 4 can be used for amplifying specific DNA fragments of the drug-resistant pathogens, but also the three primer sets formed of the DNA sequences of SEQ ID NOs. 73-78 can be used to amplify the specific DNA fragments of the wild type pathogen(s) in the sample.

It is also worth noting that the above primer sets (in Tables 3 and 4) are not limited to be used in the detection chip of the instant invention. These primer sets, besides being used for amplifying the specific DNA fragments of a patient's sample in a detection chip, these primer sets can view as one type of detection kit. As the detection kit is being used, after the specific DNA fragment of the patient's sample is amplified, the product can be placed in an appropriate detection apparatus (not limited to the detection chip), and analysis is conducted to obtain a test result.

During the synthesis of the probes, the probes with the DNA sequences as depicted in SEQ ID NOs. 1-66 are formed (step 102). The DNA sequences depicted in the SEQ ID NOs. 1-44 are formed as general mycobacterial disease probes, while the DNA sequences depicted in the SEQ ID NOs. 45 to 66 are formed as drug-resistance analysis probes for mycobacterial disease. Further, during the probe synthesis step (step 102), the 5′ ends of the DNA sequences depicted in the SEQ ID NOs. 1-66 are also modified. Consequently, these probes can covalently bind with the functional groups on the matrix surface to be immobilized on the matrix surface. The modification includes a 5′ amino modification.

Thereafter, the synthesized probes are respectively dissolved in deionized water to form a plurality of probe solutions (step 104). The probe solution has a concentration of, for example, 200 mmol/L.

A spotting step (step 106) is then conducted to spot the probe solutions respectively on the matrix. Depending on the number of spots required, the radius of the spots is about of 50 to 300 microns. Further, depending on the situation, each type of probe solutions can be spotted more than once. The surface area of the matrix is sufficiently large to accommodate tens to thousands of spots. For example, the material of the matrix is glass. Because the 5′ end of the DNA sequence has been added an amino group in the previous probe synthesis procedure (step 102), the probe solution is spotted on the matrix and is immobilized on the matrix through covalent bonding.

Thereafter, an incubation step is conducted to maintain the matrix in a moist and humid environment (step 108), wherein the incubation process is conducted at 37 degrees Celsius for three days continuously.

An oven-drying step is performed to oven-dry the matrix (step 110), wherein this oven-drying step is conducted at 80 degrees Celsius for 2 hours.

A matrix cleaning step is then conducted to clean the matrix (step 112), wherein the matrix cleaning step includes performing a cleaning process and a drying process. The cleaning solution used in the cleaning process is formed with a probe buffer solution and deionized water. The probe buffer solution is formed with 1×SSC and 0.1% of sodium dodecyl sulfate (SDS), while SSC is a solution with a pH of about 7 and is formed with 3M of NaCl and 0.3M of sodium citrate. The drying process includes, for example, blowing drying the matrix using a nitrogen gas.

A blocking step (step 114) is then conducted using a blocking solution to block the matrix surface that has not been spotted. The blocking solution used is, for example, a pH 7 solution formed with 1% bovine serum albumin (BSA) and 0.01 mol/L of phosphate buffer (PB).

A matrix cleaning step (step 116) is again conducted to clean the matrix. This matrix cleaning step includes performing a cleaning process, followed by a drying process. Further, this (second) matrix cleaning step can be repeated for several times until the matrix is completely cleaned. The cleaning solution used in this cleaning step, for example, includes deionized water to clean the excess blocking solution. The drying process is, for example, using nitrogen gas to blow dry the matrix. In one preferred embodiment, the matrix cleaning step is repeated, for example, for three times.

The fabrication of the mycobacterial disease detection chip is completed with the aforementioned method. The detection chip comprises a plurality of DNA sequences specific to mycobacterial disease. Therefore, the various pathogens of mycobacterial disease can be detected concurrently and the drug resistance of the pathogens can be verified.

It is also worth noting that during the design of the probes (step 100), a plurality of quality control probe sequences can also design. These quality control probes are synthesized and then immobilized on the matrix after the probe synthesis step and the spotting step, etc. are performed (steps 102 to 116). The quality control probes are related to the sequence of a specific substance in the test sample, which are used to ensure the sample extracted is an effective sample to prevent misjudgment of the test result.

Further, using the above method, the mycobacterial disease detection chip obtained includes a plurality of probes immobilized on the matrix. Further, each probe is selected from the group of the DNA sequences depicted in the SEQ ID NOs. 1-66. Further, each probe is formed with 15 to 25 deoxyribonucleic acids. The DNA sequences depicted in the SEQ ID NOs. 1-44 are used as probes for the typical pathogens of mycobacterial disease, while the DNA sequences depicted in the SEQ ID NOs. 45 to 66 are used as probes for the drug-resistant pathogens of mycobacterial disease. The material of the matrix is, for example, glass.

In another embodiment, each DNA sequence depicted in the SEQ ID NOs. 1-66 is immobilized on the matrix, wherein these DNA sequences are used to detect the pathogens of mycobacterial disease and their drug resistance. Further, the matrix is not only disposed with these 66 probes. Depending on the situation required, the sequences recited in SEQ ID NOs. 1-66 can be repeated for several times to constitute a microarray detection chip with tens or several thousands probes.

Since these probes are related to the various pathogens of mycobacterial disease, they can be used to determine whether the patient has contracted mycobacterial disease and the mycobacterial disease pathogens have drug resistance.

The method for detecting whether a patient has contracted mycobacterial disease using the microarray detection chip, fabricated according to the above method, is detailed in the following.

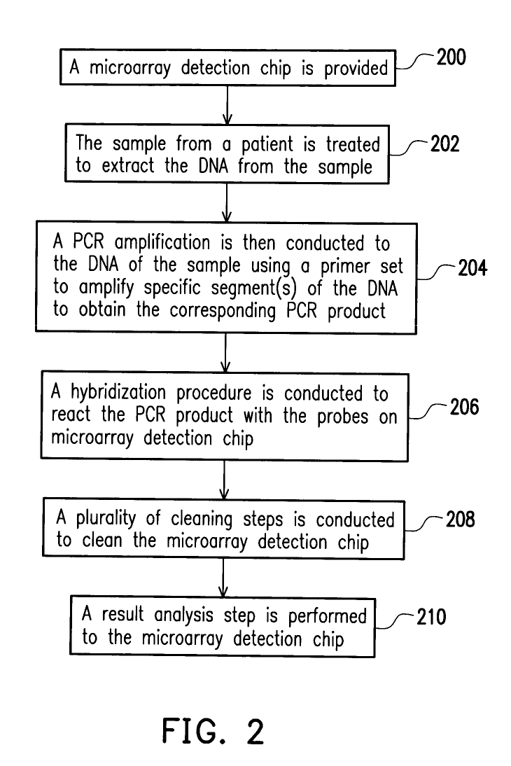

FIG. 2 is a flow diagram illustrating a method for detecting mycobacterial disease according to one embodiment of the present invention.

Referring to FIG. 2, a microarray detection chip for mycobacterial disease is provided (step 200), wherein this microarray detection chip is fabricated using, for example, the aforementioned method. Further, this microarray detection chip includes a plurality of probes specific for detecting the pathogens of mycobacterial disease. In one embodiment of the invention, besides these specific probes for detecting the pathogens of mycobacterial disease, this microarray detection chip further includes quality control probes immobilized thereon.

Thereafter, the sample from a patient is treated to extract the DNA from the sample (step 202), wherein the sample from the patient is, for example, cerebrospinal fluid, sputum, pleural fluid, ascites, paraffin-embedded tissue or excreta. If the DNA needs to be stored for a longer period time, it can be preserved at −20 degrees Celsius.

A PCR amplification is then conducted on the sample using a plurality of primer sets to amplify specific segments of the DNA to obtain the corresponding PCR product (step 204). Applied in the PCR amplification, the primer sets corresponding to the probes are selected from the 2 primer sets that are formed with the DNA sequences depicted in the SEQ ID NOs. 67-70, while the primer sets corresponding to the drug-resistance analysis probes are selected from the 4 primer sets that are formed with the DNA sequences depicted in the SEQ ID NOs. 71-78. The 2 primer sets of the DNA sequences depicted in the SEQ ID NOs. 67-70 are used as two primer sets respectively for twice PCR amplification. That is, during twice PCR amplification, the primer sets of the SEQ ID NOs. 67 and 68 are used to amplify a portion of the DNA fragments (for example, 16S-18S) and next the primer sets of the SEQ ID NOs. 69 and 70 are used to amplify fragments of the amplified DNA fragments. By doing so, the amounts of the PCR products by using twice PCR amplification are higher than those of the PCR products by using once PCR amplification.

The reagent used in the PCR amplification includes at least the DNA from the sample, DNA polymerase, at least one of the above primer sets and deoxyribonucleoside triphosphate (dNTP), wherein the DNA polymerase is, for example, Tag enzyme. Further, the PCR product is a PCR product labeled with a label. The method for labeling the PCR product includes, for example, using a labeled primer set, a labeled deoxyuridine triphosphate (dUTP) or a labeled dNTP and the above reagents to perform the PCR amplification. These labels are, for example, Cy3, Cy5 or other appropriate fluorescent materials. The label of the PCR product serves as a reference in the subsequent analysis process for determining whether the PCR product has reacted with the probe.

In the above PCR procedure, symmetric or asymmetric multiplex polymerase chain reaction is conducted. In other words, the numbers (concentration) of the forward primers and the reverse primers can be different to produce DNA with a single stranded structure in the PCR product. The single stranded DNA is subsequently hybridized with the single stranded probe on the microarray detection chip. If the numbers of the forward primer sets and the reverse primer sets are the same, a thermal denaturation procedure is conducted before the hybridization process to separate the two complementary single DNA strands before proceeding with the hybridization procedure.

The conditions for PCR amplification are shown as follow in Table 5.

TABLE 5

| STEP | Reaction Temperature | Duration Period (sec.) |
|---|---|---|
| 1 | 94 | 5 |
| 2 | 94 | 0.5 |
| 3 | 56 | 0.5 |
| 4 | 72 | 1 |
| 5 | 72 | 5 |

In this embodiment, the PCR amplification is conducted according to the conditions in step 1, followed by repeating the conditions in steps 2 to 4 for 30 cycles, and is concluded according to the conditions in step 5.

Thereafter, a hybridization procedure is conducted to react the PCR product with the probes on microarray detection chip (step 206). The hybridiation reaction is conducted, for example, in an environment of about 60 degrees Celsius for about 2 hours. Further, the hybridization reaction includes, for example, using a hybridization buffer, wherein the amount of the hybridization buffer used is the same as the amount of the PCR product. The hybridization buffer is formed with 10×SSC and 0.1% SDS. In this procedure, if the sequence of the PCR product with the single stranded structure is complementary to the sequence of the probe, the PCR product is hybridized with the probe. Further, since the PCR product comprises a label, the type of probe that is hybridized can be detected according to this label in a subsequent process.

A plurality of cleaning steps is conducted to clean the microarray detection chip (step 208), wherein the cleaning solution used in these cleaning steps is, for example deionized water. In one preferred embodiment, the microarray detection chip is repeatedly cleaned for three times. Further, with these cleaning steps (step 208), the PCR product that has not been hybridized with the probe is washed off, leaving only the PCR product that is complementary to the probe.

Thereafter, a result analysis step is performed on the microarray detection chip (step 210). The result analysis step includes performing a scanning procedure and a data analysis procedure, wherein the scanning procedure includes, for example, using a scanner to scan the mircroarray detection chip to obtain information on various test results. Further, the data analysis procedure includes, for example, using an analysis software that is compatible with the scanner to output the analysis results of the microarray detection chip. Since the PCR product after hybridization is labeled, the scanner can identify whether a label is present at the position of each probe (for example, an emission of a fluorescent signal) during the scanning step with the scanner. Therefore, based on the data analysis procedure, the type of mycobacterial disease contracted by the patient is identified and whether the mycobacterial disease pathogen is drug resistant is determined. If the microarray detection chip for mycobacterial disease shows no label besides the quality control probes at all, it is an indication that the patient does not have mycobacterial disease.

In accordance to the present invention, the fabrication of a mycobacterial disease detection chip is provided, wherein this detection chip includes a plurality of deoxyribonucleotide sequences specific to the various types of mycobacterial disease. Therefore, the detection chip is applicable to determine whether the patient has contracted mycobacterial disease and the type of mycobacterial disease.

In accordance with the present invention, the mycobacterial disease detection chip not only includes general probes for detecting mycobacterial disease, but also includes drug-resistance analysis probe for detecting whether the mycobacterial disease pathogen infected the patient is drug resistant.

The present invention also makes use of the microarray detection chip to attain a large amount and accurate analysis results.

Further, these primer sets (SEQ ID NOs. 67-78) designed according to the present invention are specific to the pathogens of mycobacterial disease. Therefore, the specific DNA fragments of the patient's sample can be amplified by using these primer sets and the subsequent testing method can be used to detect whether the patient has mycobacterial disease and the type of mycobacterial disease.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A fabrication method of a mycobacterial disease detection chip, the method comprising:
designing a plurality of probe sequences, wherein these probe sequences comprise at least DNA sequences depicted in SEQ ID NOs. (Sequence Identifier Number) 1 to 44;
performing a probe synthesis step to synthesize a plurality of probes of the deoxyribonucleotide sequences depicted in the SEQ ID NOs. (Sequence Identifier Number) 1 to 44; and
performing a spotting step to respectively spot the probes on a matrix.

2. The fabrication method of claim 1, wherein the step of designing the probe sequences further comprises designing a plurality of primer sets corresponding to the probe sequences to amplify a specific DNA fragment of a sample of a patient, wherein the primer sets comprise 2 primer sets that are formed with DNA sequences depicted in SEQ ID NOs. 67 to 70.

3. The fabrication method of claim 1, wherein the step of designing the probe sequences further comprises designing a plurality of drug-resistance analysis probe sequences, wherein the drug-resistance analysis probe sequences comprises at least DNA sequences depicted in SEQ ID NOs. 45 to 66.

4. The fabrication method of claim 3, wherein the step of designing the drug-resistance analysis probe sequences further comprises designing a plurality of primer sets corresponding to the drug-resistance analysis probe sequences to amplify a specific DNA fragment of a sample of a patient, wherein the primer sets comprise 4 primer sets that are formed with DNA sequences depicted in SEQ ID NOs. 71 to 78.

5. The fabrication method of claim 1, wherein the step of designing the probe sequences further comprises designing a plurality of quality control probes sequences.

6. The fabrication method of claim 1, wherein subsequent to the spotting procedure, the method further comprises:
performing an oven-drying step to dry the matrix; and
performing a matrix cleaning step to clean the matrix.

7. The fabrication method of claim 6, wherein subsequent to the matrix cleaning step, the method further comprises:
performing a blocking step using a blocking solution to block a matrix surface that is not spotted; and
performing another matrix cleaning step to clean the matrix.

8. The fabrication method of claim 1, wherein a spot formed by the spotting step has a radius of about 50 to 300 microns.

9. A microarray detection chip applicable for detecting mycobacterial disease, the microarray detection chip comprising:
a plurality of probes immobilized on a matrix, and the probes are selected from the group consisting of deoxyribonucleotide sequences depicted in SEQ ID NOs. (Sequence Identifier Number) 1 to 44.

10. The detection chip of claim 9, further comprising:
a plurality of drug-resistance analysis probes immobilized on a matrix, and the drug-resistance analysis probes are selected from the group consisting of deoxyribonucleotide sequences depicted in SEQ ID NOs. (Sequence Identifier Number) 45 to 66.

11. The detection chip of claim 9, further comprising a plurality of quality control probes immobilized on the matrix.

12. A mycobacterial disease detection method applicable for detecting whether a patient has mycobacterial disease, the method comprising:
providing a microarray detection chip of claim 9;
treating a sample of the patient to extract a deoxyribonucleic acid (DNA) from the sample;
using a plurality of primer sets to perform a polymerase chain reaction (PCR) on the DNA to amplify a specific fragment of the DNA to obtain a corresponding PCR product, wherein the primer sets used in the PCR are selected from the group consisting of 2 primer sets, which are formed with DNA sequences depicted in SEQ ID NOs. 67 to 70;
performing a hybridization reaction to react the PCR product with the probes on the microarray detection chip; and
performing a result analysis on the microarray detection chip.

13. The detection method of claim 12, wherein the method further comprises performing a drug resistance analysis, wherein the step of performing the drug resistance analysis comprises:
immobilizing a plurality of drug-resistance analysis probes on the microarray detection chip, wherein the drug-resistance analysis probes are selected from the group consisting of DNA sequences depicted in SEQ ID NOs. (Sequence Identifier Number) 45 to 66, and wherein a plurality of primer sets used in the PCR and corresponding to the drug-resistance analysis probes are selected from the group consisting of 4 primer sets, which are formed with DNA sequences depicted in SEQ ID NOs. 71 to 78.

14. The detection method of claim 12, wherein the microarray detection chip further comprises a plurality of quality control probes immobilized thereon.

15. The detection method of claim 12, wherein the PCR product is labeled with a label.

16. The detection method of claim 15, wherein the label comprises a fluorescent material.

17. The detection method of claim 15, wherein the labeling of the PCR product comprises using a material selected from the group consisting of a primer with the label, deoxyuridine triphosphate (dUTP) with the label and dexoyribonucleoside triphosphate (dNTP) with the label, to perform the PCR.

18. The detection method of claim 12, wherein the sample is selected from the group consisting of a cerebrospinal fluid, a sputum, a pleural fluid, an ascites, a paraffin-embedded tissue and an excreta.

19. The detection method of claim 13, wherein the microarray detection chip further comprises a plurality of quality control probes immobilized thereon.

20. A primer set for use on mycobacterial disease detection and drug resistance detection, the primer set is selected from the group of 6 primer sets that are formed with deoxyribnucleotide sequences depicted in SEQ ID NOs. 67 to 78.

21. The primer set of claim 20, wherein the primer set is a detection kit, and when the detection kit is used to amplify a specific DNA fragment of a sample of a patient, a resulting product is placed inside a detection device for analysis.
